(19) United States
(12) Patent Application Publication
(10) Pub. No.: US 2006/0034774 A1
(43) Pub. Date: Feb. 16, 2006

(54) Galactopyrannosyltriaminetetracarboxylate as ligands for bioactivated paramagnetic metal complexes

(75) Inventors: Yun-Ming Wang, Fengshan City (TW); Ya-Wen Hsu, Kaohsiung City (TW); Gin-Chung Liu, Kaohsiung City (TW)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Kaohsiung Medical University

(21) Appl. No.: 11/200,801

(22) Filed: Aug. 10, 2005

(30) Foreign Application Priority Data
Aug. 11, 2004 (TW) 093124134

(51) Int. Cl. A61K 49/10; C07H 17/00

(52) U.S. Cl. 424/9.361; 536/17.1; 536/18.7

(57) ABSTRACT

The present invention provides a triaminetetraacetatesaccharide coordinating to metal ion to form bioactive metal complexes. The bioactive metal complexes of the present invention can be used as contrast agents for magnetic resonance imaging (MRI).

### FIELD OF THE INVENTION

The present invention relates to a magnetic resonance imaging (MRI) contrast agent, and more particularly relates to a new compound of triaminetetraacetatesaccharide and its paramagnetic metal complexes used for MRI contrast agents.

### BACKGROUND OF TEE INVENTION

In recent years, due to the developments of software and hardware for magnetic resonance imaging (NM) great improvements in medical imaging diagnoses have been achieved. In order to ameliorate the sensitivity and accuracy of MRI diagnoses, developing safe and stable MRI contrast agents with targeting ability has become one of the main aims of the current MRI study. Among the metallic ions generally used for MRI contrast agents such as Mn²⁺, Fe³⁺, and Gd³⁺, Gd³⁺ is the most frequently used one because of its high magnetic moment, but the toxicity thereof is also the highest if it remains in the body. Hence it is important to make Gd³⁺ to form a stable complex by using organic ligands, so as to inhibit its toxicity.

When a new MRI contrast agent is to be designed, the stability of the metal complex is an important fact to be considered. The stability of the metal complex of a contrast agent in organisms means the stability of such a complex during the retention in an organism before being excreted out of the body. For example, the stability of the gadolinium complex in organisms depends on the three factors mentioned below: (1) the thermodynamic stability constant of the gadolinium complex, i.e. the affinity of the organic ligands to the gadolinium ions while being totally deneutronized; (2) the conditional stability constant of the gadolinium complex, i.e. the stability constant of the gadolinium complex under a physiological pH condition in organisms; and (3) the gadolinium ion selectivity constant of organic ligands (Cacheris et al., 1990, Magn. Reson. Imag., Vol. 8, p467). Because the metallic ions of Ca, Zn, Cu, and Fe existing in organisms would compete with gadolinium ions for the organic ligands, the gadolinium ions would be released from the gadolinium complexes if the gadolinium ion selectivity is inferior.

The relaxation rate of the metallic complex is another essential condition for the MRI contrast agents. Generally speaking, the factor which influences the relaxation rate is expressed as the following equation:
r₁≈q(μ_{eff})²τ_{c}/r⁶
wherein q is the number of inner space water molecules, μ_{eff} is the effective magnetic torque of the metallic ion (the μ_{eff}=0.94 Bohr Magneton for the gadolinium metallic complex, see Cotton et al., 1982, Advanced Inorganic Chemistry, Vol. 4. Ch. 23), -c is the correlation time of a paramagnetic material under a constant magnetic field, and r is the distance between the metallic ion and the proton of the inner space water molecule (r=2.50±0.04 Å for the system of Gd³⁺OH₂). For the gadolinium complexes with similar functional groups, μ_{eff} and r are regarded as constants, therefore q and τ_{c} are the major factors which influence the relaxation rate (Schauer et al., 1989, J. Chem. Soc. Dalton Trans., p 185).

The correlation time (τ_{c}) is mainly influenced by the three factors mentioned below: (1) He molecular rotational correlation time, τᵣ; (2) the electron longitudinal and transverse spin relaxation time, T_{1,2c}; and (3) the water residence lifetime or the exchange rate, τₘ⁻¹=k_{cx} (Toth et al, 2001, Coord. Chem. Rev., Vol. 216-217, p363). The relation is shown by the equation:
τ_{c}⁻¹=τᵣ⁻¹+T_{ic}⁻¹+τₘ⁻¹ i=1,2

The relaxation rate would be a maximum if the value of correlation time τ_{c} equals to the reciprocal value of the proton Larmor frequency. So it is inferred that the optimum value of τ_{c} is 7.4 ns while the magnetic field strength is 0.5 T (21 MHz ¹H frequency) and 2.5 ns while the magnetic field strength is 1.5 T (64.5 MHz). It is suggested that the longitudinal relaxation rate mainly depends on the longitudinal relaxation of bound solvent molecule, T₁ₘ and τₘ (Luz et al., 1964, Chem. Phys. Vol. 40, p2686). The relation is shown as the following equation:

$${r}_{1} = \frac{1}{{T}_{1}} = \frac{q {P}_{m}}{{T}_{1 m} + {τ}_{m}}$$

in which, Pₘ is the mole fraction of the bound solvent molecules. From the equation it would be understood that, if the exchange rate of water molecule is very fast, i.e. τₘ<<T₁ₘ, then r₁ mainly depends on the relaxation rate of the bound solvent molecule (1/T₁ₘ). Therefore, in order to achieve a higher relaxation rate, a very small value of τₘ of the gadolinium complex is usually required. However, if τₘ is too small, then T₁ₘ would be influenced by τₘ, so a higher relaxation rate is unachievable even the value of τₘ is reduced unlimitedly. Through a theoretical simulation for the influence of T_{1c}, τᵣ, and τₘ on relaxation rate, it is obtained that the optimum value of τₘ is 10 ns under the condition of simulating q=1 and r=3.1 Å while magnetic field strength is 0.5 T and 1.5 T, i.e. the most commonly used magnetic field strength in clinical (Caravan et al., 1999, Chem. Rev. Vol. 99, p2293).

Generally speaking, the augmentation of the magnetic field strength would lead to an increase of T₁ₑ. T_{1c} has a great influence on the relaxation rate in a magnetic field of 0.5 T. However, T₁ₑ does not have a very obvious influence on the relaxation rate in a magnetic field of 1.5 T. That is, the relaxation rate is only influenced by τᵣ and τₘ while the magnetic field strength is higher. The optimum value of τₘ is about 10 ns as mentioned in the previous paragraph, whereas the optimum value of τᵣ is about 20 ns. The longitudinal relaxation rates of all available MRI contrast agents on the market now are somewhat lower than their theoretical maximum values. This is mainly because the molecular rotations of these contrast agents are too fast.

The new generation of MRI contrast agents relates to a conjugation of the metal complex of small molecules (with a low molecular weight), such as [Gd(DTPA)]^{\overline{2}} or [Gd(DOTA)] (Brasch, 1991, Magn, Reson. Med., Vol. 22, p282), with something with a high molecular weight, so as to adjust their biophysical and pharmacological properties. From the view of biophysics, the molecular rotation of the contrast agent is lowered by means of the combination of the gadolinium complex of small molecules and the polymeric materials and the relaxation rate is thus increased. Besides, if the gadolinium complexes are combined with tissue-specific targeting moieties, these polymeric conjugations will bring the gadolinium complexes to receptors at low concentration by carriers, so that the receptors would be observed in MRI. Besides, because the molecules of the conjugations with high molecular weights are bigger, they would stay in vessels for a longer time, and thus they are suitable for being used in the blood pool imaging.

It is took a lot of efforts on trying to lower the molecular rotation rate of the contrast agent in order to increase the relaxation rate thereof. For example, the molecular rotation rate is lowered by substituting one of the carboxyls in the structure of 1,4,7,10-tetrakis(carboxymethyl) -1,4,7,10-tetraazacyclododecane (DOTA) with huge functional groups containing benzenes or more carboxyls and following by combining with Gd³⁺ to form the metal complex, of which the values of r, are 4.03, 4.33, 4.99, and 5.19 mM⁻¹s⁻¹ respectively, all of which are higher than that of [Gd(DOTA)]⁻, 3.56 mM⁻¹s⁻¹. Because of the increase of the value of τᵣ, the value of r₁ climbs according to the increase of the molecular weights of the organic ligands (Lauffer et al., 1987, Chem. Rev., Vol. 87, p901; Aime et al., 1992, Inorg. Chem., Vol. 31, p2422).

There are many methods developed for linking the gadolinium complex to a high molecular weight material (Brinkley, 1992, Bioconjugate Chem., Vol. 3, p2), wherein the acylation, the alkylation, the formation of ureas, and the reduction of amination are popular applied. The most frequently used reagent to be combined with a high molecular weight material includes DTPA, the derivatives thereof, and DTPA-dianhydride. By the reaction of the primary amine in the high molecular weight material with DTPA, the derivatives thereof, or DTPA-dianhydride, the organic ligands would be combined therewith. Sieving et al. (1990, Bioconjugate Chem., Vol. 1, p65) disclose the reaction of Polylysines with variant molecular weights with DTPA-dianhydride and the derivatives of DTPA. However, the cross-linking always happens very easily during the combination of DTPA-dianhydrare and proteins. So, by the reaction of N-hydroxysuccinimide with DTPA to form a N-hydroxysuccinic ester, the crossing-linking is evitable (Spanoghe et al., 1992, Magn, Reson. Imaging, Vol. 10, p913). Paxton et al. (1985, Cancer Res., Vol. 45, p5694) disclose that [DTPA-(N-hydroxysuccinic ester)] would form a covalent binding not only with the protein but also with the monoclonal antibody, and by this carrier the contrast agent would be brought to the anticarcinoembryonic antigen. However, this synthesis method also leads to the production of peptide bonds, so that the binding ability of this organic ligand to the gadolinium complex is weakened. In order to overcome this problem, Aime et al. (1999, Bioconjugate Chem., Vol. 10, p192) disclose a method in which the covalent binding is formed between 1,4,7-trikis(carboxymethyl)-1,4,7,10-tetraazacyclododecane (DO3A) and proteins with a linker of 3,4-diethoxycyclobut-3-ene-1,2-dione,squarate. Through his method, the stability of the metal complex is enhanced, and the relaxation rate of the gadolinium complex is also increased due to the augment of the molecular weight. In addition, a specific bioactivity is achieved via the formation of the covalent binding with the protein.

Right now, the MRI contrast agents approved by FDA for clinical usage in intravenous injection include [Gd(DTPA)]²⁻, gadoterate megulumine ([Gd(DOTA)]⁻), [Gd(DTPA-BMA)] (bis-methylamaide gadodiamide injection), 1,4,7-trikis (carboxymethyl)-10-(2-hydroxylpropane, -1,4,7,10-tetraazacyclododecane, [Gd(HP-DO3A)]gadoteridol), and the five extracellular agents of Manganese, dipyridoxyl, diphosphate, MnDPDP, and Teslascan. Among the mentioned agents, [Gd(DTPA-BMA)] and [Gd(HP-DO3A)] are nonionic contrast agents while [Gd(DTPA)]²⁻, [Gd(DOTA)]⁻, and MnDPDP are ionic contrast agents. Moreover, [Gd(DOTA)]⁻ and [Gd(HP-DO3A)] are macrocyclic, and MnDPDP, [Gd(DTPA)²⁻] and [Gd(DTPA-BMA)] are open-chained.

Because the concentration at which MRI contrast agent is capable of targeting lesions (such as receptors or antigens) falls in a nanomolar level, which is too low for the receptor-induced magnetization enhancement (RIME) to be used in MRI, during the recent years scientists try to use the enzyme to activate the gadolinium complex, so as to increase the concentration of the contrast agent at the targeted location approximately. Through the method, the relaxation rate of the metal complex is increased, and the target-to-background ratio is also enhanced. Furthermore, 4,7,10-tri(aceticacid)-1-(2-β-galactopyranosylethoxy)-1,4,7,10-tetraagacyclo dodecane) (gadolinium III, Egad) is synthesized by connecting one of the functional groups to the macrocyclic organic ligand (Moats et al., 1997, Chem. Int. Engl., Vol. 36, p726). In this case, because Egad is combined with the gadolinium ion in the form of 9-coordinate, the number of inner space water molecules of Egad is 0.7. That is to say, when Egad enters the organism and meets the β-galactosidase (β-gal), the o-nitrophenyl-β-galactopyranoside in the Egad would be hydrolyzed and removed, so as to be bound with 1.2 inner space water molecules, and the MRI signals are thus improved.

The Pro-RIME with an increased receptor-induced magnetization is also synthesized (Nivorozhkin et al., 2001, Angew. Chem. Int. Ed., Vol. 15, p2903). This reagent is composed of: (1) a masking group consisting of three lysine residues, (2) an HAS binding site, (3) a glycine linker, and (4) a signal generation group. The mechanism of action involves that the degradation of lysines in the outermost space is easily achieved by the human carboxypeptidase B thrombin-activatable fibrinolysis inhibitor (TAFI). Once the degradation of the three lysine residues is completed, the exposed lipid soluble aromatics would produce a great binding ability with HSA, and thus a higher relaxation rate is achieved.

A derivative of DTPA, 3,6,10-tri-(carboxymethyl)-3,6,10-triazadodecanedioic acid (TTDA), is also synthesized and the physical and chemical properties of the metal complexes of Gd³⁺, Zn²⁴, Ca²⁺, and Cu²⁺ formed therewith are also studied in detail. The studies show that [Gd(TTDA)]²⁻ has better physical and chemical properties than [Gd(DTPA)]²⁻, so it has a great potential to be an MRI contrast agent (Wang et al., 1998, J. Chem Soc. Dalton Trans., p4113-4118).

Based on the above, gadolinium complexes with a high stability and a high relaxation rate are the emphases of research during the recent years. It is important and useful to find better NM contrast agents. Hence the present invention provides new bioactive metal complexes with a potentiality and a high stability as MRI contrast agents.

### SUMMARY OF THE INVENTION

The present invention relates to a magnetic resonance imaging (MRI) contrast agent, and more particularly to a new compound of triaminetetraacetatesaccharide and its paramagnetic metal complexes used for MRI contrast agents.

It is an aspect of the present invention to provide a new triaminetetraacetatesaccharide compounds, preferably galactopyrannosyl-triaminetetracarboxylate.

It is another aspect of the present invention to provide a kind of paramagnetic metal complex, which is used as a MRI contrast agent.

In the present invention, it is found that the gadolinium complexes of TTDA and their derivatives have high inner space water molecule exchange rate, and their τₘ values are close to their theoretical optimum values for reaching to the highest r₁. Although this property has no obvious influence on the relaxation rates of gadolinium complexes of small molecular weight, however, by combining the gadolinium complexes of TTDA with high molecular weight materials (such as proteins), such a property of high inner water molecule exchange rate owned by gadolinium complexes of TTDA contributes a lot to the increase of relaxation rate with the augmentation of the molecular weight and the reduction of the correlation time of its molecular rotation. One aspect of the present invention is the synthesis of a ligand for bioactive gadolinium complexes by combining TTDA with o-nitrophenyl-β-galactopyranoside.

According to the aspect of the present invention, a new triaminetetraacetatesaccharide compound having the following formula has be synthesized: 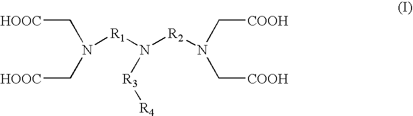
wherein R₁ is —(CH₂)ₐ— or —(CH₂)ₐ—X—(CH₂)ₐ, a is a first integer from 2 to 4, R₂ is —(CH₂)_{b}— or —(CH₂)_{b}—X—(CH₂)_{b}—, b is a second integer from 2 to 4, R₃ is —(CH₂)_{c}— or —(CH₂)_{c}—X—(CH₂)_{c}—, c is a third integer from 2 to 4, X is —O— or —S—, and R₄ is one selected from a group consisting of a galactopyranose, a monosaccharide, and a polysaccharide.

The present invention also provides a kind of paramagnetic metal complex for being an MRI contrast agent with chemical structure of ML, wherein M is a central metallic ion being one selected from a group consisting of ions of Zn, Fe, Co, Cu, Ni, and Cr, and L is an organic ligand which is a compound having the chemical formula (I): 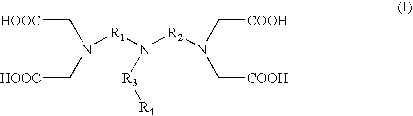
wherein R₁ is —(CH₂)ₐ— or —(CH₂)ₐ—X—(CH₂)ₐ—, a is a first integer from 2 to 4, R₂ is —(CH₂)_{b}— or —(CH₂)_{b}—X—(CH₂)1—, b is a second integer from 2 to 4, R₃ is —(CH₂)_{c}— or —(CH₂)_{c}—X—(CH₂)_{c}—, c is a third integer from 2 to 4, X is —O— or —S—, and R₄ is one selected from a group consisting of a galactopyranose, a monosaccharide, and a polysaccharide.

Preferably, the 3-amine-4-caboxyl-o-nitrophenyl-β-galactopyranoside compounds having the chemical formula (I) are also provided; 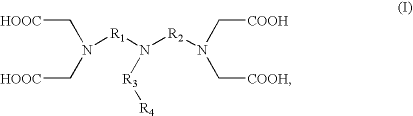
wherein R₁ is —(CH₂)ₙ, n is 2, R₂ is —(CH₂)ₘ, m is 3, R₃ is —(CH₂)ₓ, x is 2, and R₄ is a galactopyranose.

The mentioned 3-amine-4-caboxyl-o-nitrophenyl-β-galactopyranoside compounds synthesized according to the present invention have the following properties:
1. a higher selectivity with the gadolinium complex,
2. the ability for increasing the concentration of the contrast agent at targeted location by using the enzyme to activate the gadolinium complex, and
3. the ability for forming the targeted gadolinium complex by the galactopyranose contained herein.

The triaminetetraacetatesaccharide compounds according to the present invention are used as ligands to coordinate with metallic ions to form paramagnetic metal complexes. These paramagnetic metal complexes are used as MRI contrast agents and have chemical structures of ML, wherein M is a central metallic ion being one selected from a group consisting of ions of Zn, Fe, Co, Cu, Ni, and Cr, and L is an organic ligand having the chemical formula (1) mentioned above.

Preferably, the metallic ions are Gd³⁺, Fe³⁺, and Zn²⁺.

The above aspects and advantages of the present invention will become more readily apparent to those ordinarily skilled in the art after reviewing the following detailed description and accompanying drawings.

### BRIEF DESCRIPTION OP THE DRAWINGS

FIG. 1 is a schematic view illustrating the pathway of the synthesis of CGP according to a preferred embodiment of the present invention.

FIG. 2 is a schematic view illustrating the conversion of [Gd(CGP)]⁻ to [Gd(CHE)]⁻ according to a preferred embodiment of the present invention.

FIG. 3 is a diagram illustrating the displacement of Dy (III)-induced H₂O ¹⁷O-NMR with respect to the concentrations of DyCl₃ and [Dy(CGP)]⁻ according to a preferred embodiment of the present invention.

FIG. 4 is a diagram showing the result of HPLC test obtained on the 5^{th} day after adding the enzyme into the [Gd(CGP)]⁻ according to a preferred embodiment of the present invention.

FIGS. 5(a) and 5(b) are diagrams showing the respective MRI images of [Gd(CGP)]⁻ with and without the enzyme according to a preferred embodiment of the present invention.

### DETAILED DESCRIPTION OF THE INVENTION

The present invention is described more specifically with reference to the following embodiments. It is to be noted that the following descriptions of preferred embodiments of this invention are presented herein for the purpose of illustration and description only; it is not intended to be exhaustive or to be limited to the precise form disclosed.

The Evaluation of Bioactive Paramagnetic Metal Complexes

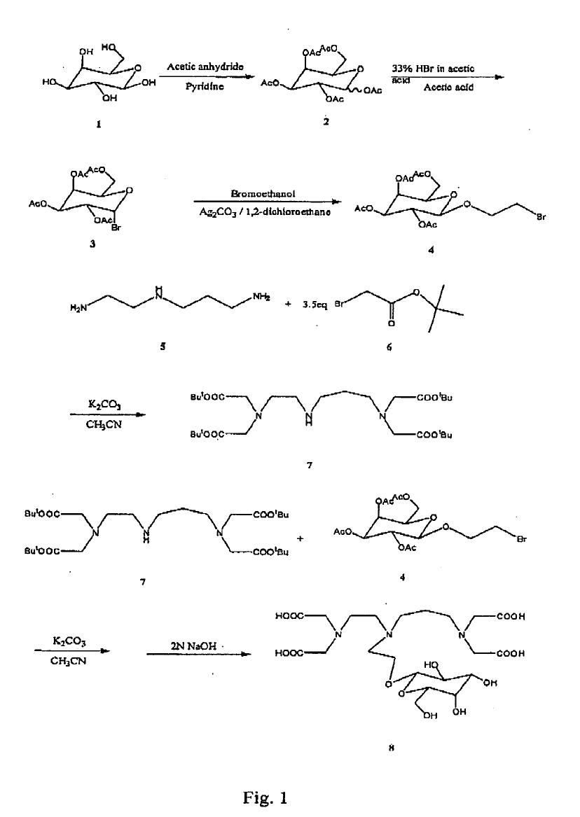

According to the present invention, the synthesis method of triaminetetraacetatesaccharide compounds with the chemical formula of (I) is described as the following steps shown in FIG. 1.

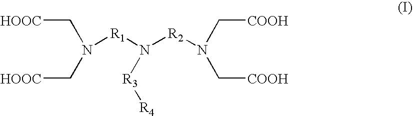

0.11 mole of D-galactose, Compound 1 shown in FIG. 1, and anhydrous pyridine are put together into a single neck flask. Then acetic anhydride is slowly added through the isobaric valve, and the mixture is stirred at room temperature. After the reaction, trichloromethane is added, and the organic layer is extracted and collected by using solutions such as deionized water in proper order. The organic layer is filtered by adding sodium sulfate, and the product, 1,2,3,4,6-pentaacetate-D-galactose, Compound 2 shown in FIG. 1, is obtained.

Then 0.06 mole of 1,2,3,4,6-pentaacetate-D-galactose, Compound 2, is put into a single neck flask together with glacial acetic acid. HBr is slowly added through the isobaric valve, and stirred at room temperature. After the reaction the chloroform solution is added, and the organic layer is extracted and collected by using solutions such as deionized water in proper order. The sodium sulphate is added into the organic layer. After being filtered, dried, and purified by the silica gel column chromatography, the product 2,3,4,6-aceto-α-D-bromogalacopyranose, Compound 3 shown in FIG. 1, is collected.

2.32×10⁻³ mole of 2,3,4,6-aceto-α-D-bromogalacopyranose, Compound 3, and the molecular sieve of 4 Å are put together into a three-neck flask. The air is pumped out to form vacuum, and then nitrogen gas is filled. An adequate amount of dicholoethane is added as the solvent, and then 2-bromoethanol is provided therein. The solution is stirred for five minutes, and then silver carbonate is provided therein. After the reaction at room temperature is completed, the solution is filtered and purified by the silica gel column chromatography. Thus, the product 2,3,4,6-aceto-1-ethylbromogalacopyranose, Compound 4 shown in FIG. 1, is collected.

Another chemical, 0.171 mole of N-aminoethyl-1,3-propanediamaine, Compound 5 shown in FIG. 1, is put in the single neck flask. After acetonitrile is added, the solution is fully mixed. Then potassium carbonate is provided therein, and the solution is stirred at room temperature. After tert-butyl bromnoacetate, Compound 6 shown in FIG. 1, is added, the solution is heated and circulated for reacting. Then the solution is filtered and extracted by solutions such as deionized water in proper order. Thus, the organic layer is collected. After being dried, the yellow oil-like material, 3,10-di(carboxymethyl)-3,6,10-triazadodecanedioic(tetra)-tert-butyl ester, Compound 7 shown in FIG. 1, is yield.

3.13×10⁻³ mole of 3,10-di(carboxymethyl)-3,6,10-triazadodecanedioic(tetra)-tert-butyl ester, Compound 7, is put into the single neck flask and dissolving in the acetonitrile. Then tetramethylguanidine is added and stirred. Meanwhile, 2,3,4,6-aceto -1-ethylbromo galacopyranose, Compound 4, is provided and heated. The solution is removed and extracted, and the organic layer therein is collected and purified by the silica gel column chromatography. The yellow oil-like material obtained in the previous step is put into the round-bottomed flask, and 2N NaOH is also provided therein. After being stirred, heated, and purified with the anion exchange resin, 6-((β-galactopyranosyloxy)ethyl)-3,10-di(carboxymethyl)-3,6,10-triazado decanedioic acid (CGP. Compound 8 shown in FIG. 1) is obtained.

0.18 m mole of COP is mixed with 0.27 m mole of gadolinium chloride, and the deionized water is provided. After being heated, the pH value of the solution is adjusted step by step. Then, after filtering and removing the solution, the colorless crystals of [Dy(CGP)]⁻ is precipitated.

In this embodiment, the mentioned process of “extract” means to extract the solution through the deionized water and ethyl acetate, through the deionized water and trichloromethanal, or through deionized water, sodium bicarbonate, and saturated salt solution, which depends on the properties of the product to be extracted. Besides, the pH value of the solution is adjusted step by step means that the pH value is adjusted to reach 8 so that the solid would precipitate first, and then the pH value of the solution is adjusted to 7.

According to the present invention, the provided bioactive metal complex is formed by the ligand, triaminetetraacetatesaccharide, and the coordinated metallic ions. These kinds of bioactive metal complexes can be used as MRI contrast agents.

Discussion of the number of inner space water molecule:

From measuring the d.i.s. (Dy III-induced ¹⁷O-NMR water shifts) of the ¹⁷O nuclides in water induced by Dy III ions via ¹⁷O-NMR and graphing the concentration of Dy III complexes with respect to the d.i.s., a linear relationship is found as shown in the following equation:
d.i.s.=qΔ[Dy(ligand)ₙ(H₂O)_{q}/[H₂O]

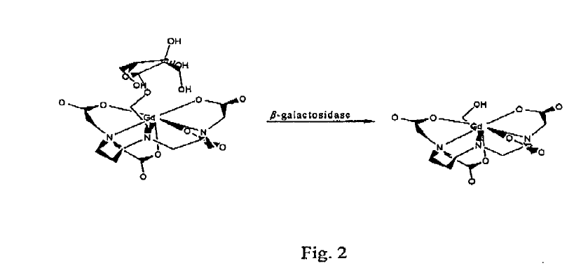

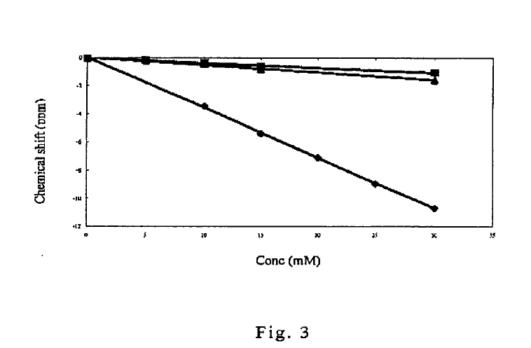

The slope is qΔ/[H₂O] with q being the number of inner space water molecule. Referring to FIG. 2, it illustrates the conversion of [Gd(CGP)]⁻ to [Gd(CHE)]⁻ in the present of β-galactosidase (D-gal) according to the present invention. In FIG. 3, the slopes of Dy(CGP)— alone and the one added with β-galactosidase for 14 days are −35.6 ppm/mM (r²=0.9981) and −54.6 ppm/mM (r²=0.9968) respectively, while the slope of Dy III induced ¹⁷O is −358.1 ppm/mM (r²=0.9998). Because Day III hydrate is combined with 8 water molecules and is in direct proportion to the slope, it shows that the values of q obtained from [Gd(CGP)]⁻ which fails to react with β-galactosidase and from the one which is added with β-galactosidase for 14 days are 0.8 and 1.2 respectively. Because of the increase of the value of q, the MRI signals is accordingly fortified as shown in FIG. 3.

The analysis of HPLC:

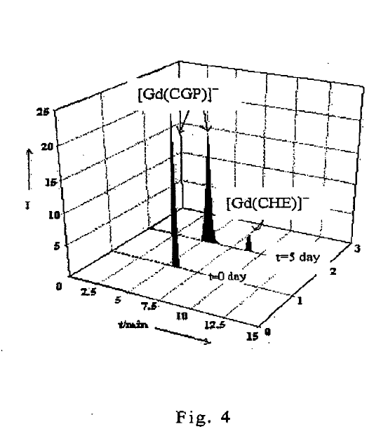

β-galactosidase is provided to [Gd(CGP)]⁻ and reacts therewith at 37±0.1° C. The [Gd(CGP)]⁻ is analyzed before and after the enzyme was added on the 0^{th} day and 5^{th} day respectively. The HPLC maps obtained at different times are shown in FIG. 4.

In the map, the x-axis shows the retention time, and the y-axis indicates the intensity. It discloses that, at the time t=0 day, there is a peak with the retention time being about 6 minutes, and this peak represents the [Gd(CGP)]⁻ before the reaction with β-galactosidase. When the time t=S days, there is still a peak with retention time being about 6 minutes, but the intensity is obviously lower. There is another peak which appears with the retention time being about 9 minutes, and this peak represents the gadolinium complex from the reaction with β-galactosidase, in which the β-galactosidase on [Gd(CGP)]⁻ is removed. The gadolinium complex is [Gd(CHE)]⁻.

The research of relaxation time:

Three samples of [Gd(CGP)]⁻ react at 37±0.1° C. with β-galactosidase at different concentrations. The change of T₁ is measured by nuclear magnetic spectrometer of 400 MHz at 25±0.1° C. at different times. With the longitudinal relaxation time of [Gd(CGP)]⁻ before the β-galactosidase was added as denominator and the longitudinal relaxation time of the samples as numerator, there is the percentage of the change of T₁.

The following table shows He percentage of the change of T₁ of three [Gd(CGP)]⁻ samples at different concentrations and different times:

| | immediately after | after reaction |
|---|---|---|
| samples | added with the enzyme | for 14 days |
| [Gd(CGP)]⁻ + 2.4 nM β-gal | 95.74 ± 1.47% | 85.91 ± 0.11% |
| [Gd(CGP)]⁻ + 7.2 nM β-gal | 93.26 ± 0.25% | 76.63 ± 1.11% |
| [Gd(CGP)]⁻ + 7.2 nM heat- | 99.60 ± 1.78% | 102.72 ± 0.58% |
| inactivated β-gal | | |

From aforesaid table, it is found that if the values of T₁ of the three samples are measured after adding the enzyme, the values of T₁ are almost constant. But if the values of T₁ are measured after 14 days, it is found that the value of T₁ of [Gd(CGP)]⁻ with lower concentration of enzyme (2.4 nM) decreases by about 15%, the value of T₁ of [Gd(CGP)]⁻ with higher concentration of enzyme (7.2 nM) decreases by about 25%, and the value of T₁ of [Gd(CGP)]⁻ in the other control group with higher concentration of enzyme (7.2 nM) and reaction at 80° C. for 30 minutes (to deactivate the enzyme) is almost constant. From the results, it is found that the higher the concentration of the enzyme added in and the longer the time of reaction, the more the β-galactosidases are cut, thus, the more the value of T₁ decreases. Moreover, the MRI signals is also fortified because of the decrease of the value of T₁.

The research of MRI:

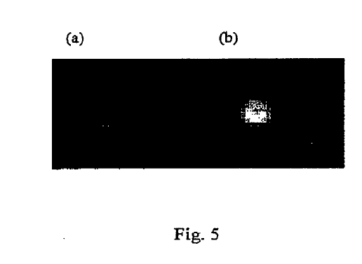

The MRI images of 0.6 mM [Gd(CGP)]⁻ before and after the reaction for 14 days at 37.0±0.1° C. with pH 7.3 7.2 nM enzyme solution mixed with 0.1 M Tris buffer solution are compared with each other. As shown in FIGS. 5(a) and 5(b), which shows the respective MRI images of [Gd(CGP)]⁻ with and without the enzyme according to a preferred embodiment of the present invention, the image before the reaction with enzyme is darker (FIG. 5(a)), while the signals after the reaction with enzyme in the image are obviously fortified (FIG. 5(b)). This result shows that [Gd(CGP)]⁻ is a bioactive (enzyme active) MRI contrast agent.

There are several preferred embodiments below. They are just the illustrations of the methods, the characteristics, and the advantages according to the present invention.

### The Preferred Embodiment I: The Synthesis of the Ligand of Triaminetetraacetatesaccharide Compound

### EXAMPLE 1

### The Synthesis Method of 1,2,3,4,6-pentaacetate-D-galactose, Compound 2

20 g (0.11 mole) of D-galactose, Compound 1, and 100 ml of anhydrous pyridine are put into a single neck flask, and 50 ml of acetic anhydride is added through the isobaric valve. Then the mixture is stirred for 8 hours at room temperature. After the reaction, the mixture is dried out, and 300 ml of chloroform is added therein. The organic layer is extracted and collected by using deionized water, sodium bicarbonate, and saturated saline in proper order. 5 g of sodium sulphate is added into the organic layer and filtered after 30 minutes, After being dried, 34.23 g of the product is obtained with a yield value of 79% (no further purification).

### EXAMPLE 2

### The Synthesis Method of 2,3,4,6-aceto-α-D-bromogalacopyranose, Compound 3

23.4 g (0.06 mole) of 1,2,3,4,6-pentaacetate-D-galactose, Compound 2, and 100 ml of glacial acetic acid are put into a single neck flask. The single neck flask is wrapped with the aluminum foil to keep it away from the light. 120 ml of 33% HBr are added through the isobaric valve slowly and stirred for 8 hours at room temperature. 800 ml of chloroform is added after the reaction. The organic layer is extracted and collected by using deionized water, sodium bicarbonate, and saturated saline in proper order. 10 g of sodium sulphate is added and filtered after 30 minutes. After being dried, the mixture is purified by the silica gel column chromatography (ethyl acetate:hexane=2:1), and collected at point 3 by TLC. After being dried, 16.56 g of the product is obtained with a yield value of 67.18%, in which the ¹H NMR (200 MHz, CDCl3): δ=6.65 (d, 1H), 5.46 (d, 1H), 5.35 (dd, 1H), 4.99 (dd, 1H), 4.44 (t, 1H), 4.10 (m, 2H), 2.15 (s, 3H), 2.09 (s, 3H), 2.00 (s, 3H), 1.97 (s, 3H), and the ¹³C NMR (50 MHz, CDCl3): δ=20.37, 20.41, 20.53, 60.70, 66.93, 67.69, 67.90, 71.00, 88.06, 169.5, 169.6, 169.8, 170.0.

### EXAMPLE 3

### The Synthesis Method of 2,3,4,6-aceto-1-ethylbromogalacopyranose, Compound 4

9.56 g (2.32×10⁻³ mole) of 2,3,4,6-aceto-α-D-bromogalacopyranose, Compound 3, and 0.5 g of the molecular sieve of 4 Å are put into the three-neck flask. The air is pumped out to form vacuum, and then nitrogen gas is filled. An adequate amount of dicholoethane is added as the solvent, and then 3.466 ml (4.88×10⁻³ mole) of 2-bromoethanol is provided therein. The solution is stirred for five minutes, and then 4.6 g (2.32×10⁻³ mole) of silver carbonate is added therein. The reaction is preceded for 2.5 hours at room temperature, and the air is pumped out after the reaction is completed. The solution is filtered and purified by the silica gel column chromatography (ethyl acetate:hexane=1:2). The product is collected at point 2 by TLC. After being dried, 3.12 g of product is obtained with a yield value of 29.49%, in which the ¹H NMR (200 MHz, CDCl₃): δ=5.40 (d, 1H), 5.19 (dd, 1H), 5.06 (dd, 1H), 4.60 (d, 1H), 4.17 (m, 4H), 4.00 (t, 1H), 3.91 (m, 1H), 3.51 (t, 2H), 2.16 (s, 3H), 2.08 (s, 3H), 2.05 (s, 3H), 1.98 (s, 3H), and the ¹³C NMR (50 MHz, CDCl₃) δ=20.04, 20.09, 20.12, 20.31, 60.82, 66.59, 68.09, 69.19, 70.23, 70.27, 100.8, 168.9, 169.5, 169.6, 169.7.

### EXAMPLE 4

### The Synthesis Method of 3,10-di(carboxymethyl)-3,6,10-triazadodecanedioic(tetra)-tert-butyl ester, Compound 7

20 g (0.171 mole) of N-aminoethyl-1,3-propanediamine, Compound 5, is put into a single neck flask, and 100 ml of acetonitrile is added The solution is rally mixed. Then 25 g of potassium carbonate is added, and the solution is stirred for an hour at room temperature. After 88.20 ml (0.149 m mole) of tert-butyl bromoacetate, Compound 6, is added, the solution is heated and circulated for 24 hours. After the reaction is completed, the filtrate is filtered and pumped out. The dried oil-like material is extracted by using 200 ml deionized water and 100 ml of chloroform, and the organic layer is collected. After being dried, an oil-like material is obtained. The oil-like material is purified by the silica get column chromatography (ethyl acetate→ethyl acetate:acetone=1:1). The portion, in which ethyl acetate:acetate=1:1, is collected. After being dried, a yellow oil-like material is obtained. Then the oil-like material is dissolved by adding an adequate amount of ethyl acetate and put at room temperature for recrystallization. After three days, there are 29.32 g of transparent crystals precipitated with a crystallization value of 30%, and the melting point is 105.1-106.4 degrees, in which the ¹H-NMR (CDCl₃, 400 MHz): δ=3.56(s, 4H), 3.51(s, 4H), 3.27 (t, 2H), 3.22(t, 2H), 3.06 (t, 2H), 2.90(t, 2H), 2.07(p, 2H), 1.45(s, 36H), and the ¹³C-NMR (CDCl₃, 100 MHz): δ=171.1, 170.8, 81.6, 81.3, 55.2, 54.9, 52.1, 50.3, 49.2, 45.9, 28.1, 22.9.

### EXAMPLE 5

### Preparation of 6-((β-galactopyranosyloxy) ethyl)-3,10-di(carboxymethyl)-3,6,10-tiazadodecanedioic acid, Compound 8 (COP)

1.8 g (3.13×10⁻³ mole) of 3,10-di(carboxymethyl)-3,6,10-triaza dodecanedioic(tetra)-tert-butyl ester, Compound 7, is put into a single neck flask and dissolving in acetonitrile. 3.962 ml (3.13×10⁻² mole) of tetramethylguanidine is added, and the solution is stirred for 10 minutes. 1.71 g (3.76×10⁻³ mole) of 2,3,4,6-aceto-1-ethylbromo galacopyranose, Compound 4, is added. The solution is heated and circulated for 24 hours. After drying the solution, the organic layer is extracted and collected by using the deionized water and ethyl acetate. After being dried, the solution is purified by the silica gel column chromatography (acetone:hexane=3:7→acetone:hexane=1:1), and the portion, which acetone:hexane=1:1, is collected. After being dried, a yellow oil-like material is obtained. The oil-like material is put into a round-bottomed flask, and 100 ml 2N NaOH is added therein. The solution is heated up to 40 degrees and stirred for 24 hours. The solution is purified by the anion exchange resin and eluted with the formic acid at different concentrations. The formic acid elutant of 0.04 N˜0.05 N is collected. After being dried, 0.47 g of product is obtained with a yield value of 45.06%, in which the ¹H NMR (400 MHz, CDCl₃): 6-4.31 (d, 1H), 4.18 (br, 1H), 3.95 (br, 1H), 3.81 (s, 4H), 3.80 (s, 1H), 3.63 (s, 4H), 3.62 (s, 1H), 3.45 (m, 8H), 3.31 (m, 6H), 2.11 (br, 2H), and the ¹³C NMR (100 MHz, CDCl₃): δ=18.97, 49.61, 50.49, 51.14, 52.84, 52.91, 56.39, 56.90, 60.98, 62.97, 68.53, 70.51, 72.55, 75.21, 102.6, 169.8, 173.2.

### The Preferred Embodiment II: The Synthesis of Gadolinium Complex

The Preparation of [Gd(CGP)]⁻

0.1 g (0.18 m mole) of COP (acquired in Example 5) is mixed with 1.26 g (0.27 m mole) of gadolinium chloride, and then 5 ml of deionized water is added. The mixture is heated and circulated for 24 hours. After the reaction, the pH value is adjusted to 8 so that the solids would precipitate. Then the pH value is adjusted to 7, and the solution is filtered. After removing the solution, 0.073 g of transparent crystals is precipitated with a yield value of 57.03%.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A compound having a formula I: 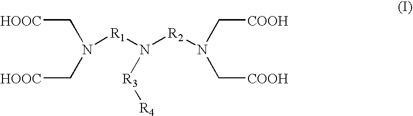
wherein R₁ is —(CH₂)ₐ— or —(CH₂)ₐ—X—(CH₂)ₐ—, a is a first integer from 2 to 4, R₂ is —(CH₂)_{b}— or —(CH₂)_{b}—X—CH₂)_{b}—, b is a second integer from 2 to 4, R₃ is —(CH₂)_{c}— or —(CH₂)_{c}—X—(CH₂)_{c}—, c is a third integer from 2 to 4, X is —O— or —S—, and R₄ is one selected from a group consisting of a galactopyranose, a monosaccharide, and a polysaccharide.

2. A compound having a formula I: 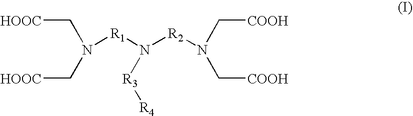
wherein R₁ is —(CH₂)ₙ, n is 2, R₂ is —(CH₂)ₘ, m is 3, R₃ is —(CH₂)ₓ, x is 2, and R₄ is a galactopyranose.

3. A metal complex, comprising a structure of ML,
wherein M is a central metallic ion being one selected from a group consisting of ions of Zn, Fe, Co, Cu, Ni, and Cr, and
L is an organic ligand having a formula I: 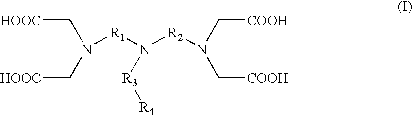
wherein R₁ is —(CH₂)ₐ— or —(CH₂)ₐ—X—(CH₂)ₐ—, a is a first integer from 2 to 4, R₂ is —(CH₂)_{b}—or —(CH₂)_{c}—X—(CH₂)_{b}—, b is a second integer from 2 to 4, R₃ is —(CH₂)_{c}— or —(CH₂)_{c}—X—(CH₂)_{c}—, c is a third integer from 2 to 4, X is —O— or —S—, and R₄ is one selected from a group consisting of a galactopyranose, a monosaccharide, and a polysaccharide.

4. A metal complex, comprising a structure of ML,
wherein M is a central metallic ion being one selected from a group consisting of ions of Zn, Fe, Co, Cu, Ni, and Cr, and
L is an organic ligand having a formula I: 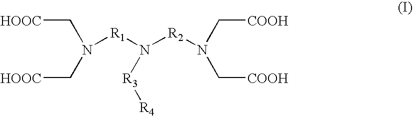
wherein R₁ is —(CH₂)ₙ, n is 2, R₂ is —(CH₂)ₘ, m is 3, R₃ is —(CH₂)ₓ, x is 2, and R₄ is a galactopyranose.
